(19) United States
(12) Patent Application Publication
(10) Pub. No.: US 2007/0073250 A1
(43) Pub. Date: Mar. 29, 2007

(54) Implantable port

(76) Inventor: James A. Schneiter, Lake Forest, IL (US)

Correspondence Address: BRINKS HOFER GILSON & LIONE, P.O. BOX 10395, CHICAGO, IL, 60610, US

(21) Appl. No.: 11/177,932

(22) Filed: Jul. 8, 2005

(51) Int. Cl. A61M 31/00; A61M 37/00; A61B 19/00

(52) U.S. Cl. 604/288.01; 604/502; 128/898

(57) ABSTRACT

An implantable port for use in the withdrawal and/or delivery of a fluid to a patient is provided. The port has a body portion having a generally curved shaped reservoir. A septum extends across the reservoir and a stem extends from the reservoir to an external position. A fluid passageway is formed from the reservoir through the stem adjacent the septum.

### FIELD OF THE INVENTION

The present invention relates generally to the field of implantable ports for use in the delivery and/or removal of a fluid to or from a patient. More specifically, the present invention relates to an implantable port having improved fluid flow and flushing effectiveness.

### BACKGROUND

Implantable access ports were initially developed to solve many of the problems created in patients with limited peripheral access combined with the need for frequent venipuncture. Prior to the development of implantable access ports, many oncology patients in need of long-term, intensive therapy could not receive a full treatment cycle of chemotherapy due to the loss of peripheral access sites on the patient. First introduced in 1983, implantable access ports have become a standard of use in the treatment of patients with oncological diseases. In addition to oncology patients, implantable access ports are used today for a wide variety of patient access options including the peritoneal cavity, the pleural cavity and access to the venous and arterial systems.

Various medical procedures require the use of an implantable access port for the delivery and/or removal of a fluid to or from a patient. One example is the infusion of a cytotoxic drug (5FU, Taxol, etc.) into the vascular system of a cancer patient, e.g., chemotherapy. Other examples include but are not limited to the infusion of blood products, antibiotics, hydration, blood sampling, aspirating ascites effusions, aspirating pleural cavity effusions, therapeutics, bone marrow transplantation (BMT) and total parenteral nutrition (TPN). When functioning properly, implantable ports are vital for patients when peripheral access is no longer an option for their care.

Implantable access ports have proven to be very beneficial in patients with a long-term need for frequent access to deliver a wide range of therapies. However, many problems have been associated with their use. The most frequently documented problems with ports used for vascular access are occlusions and infections. Clinical studies over the past fifteen years have consistently shown significant occlusion rates. The inability to withdraw blood from a port (“partial withdrawal occlusion”) or the complete inability to withdraw blood or infuse fluids (“total occlusion”) can be caused by a buildup of thrombused blood and/or drug residuals within the reservoir of the port or within the associated catheter itself. Although rare, occlusions can also be caused by a fibrin sheath that forms on the distal end of the catheter tip.

The buildup of thrombused blood and/or drug residuals (“sludge”) within the reservoir of vascular access ports was first documented in a 1991 article entitled “Partial Occlusion of Indwelling Central Venous Catheters” (Journal of Intravenous Nursing, Vol. 14, No. 3 May/June 1991). Upon examination of the ports “[1]t was noted that the catheter connection site in each port varied in distance from the floor of the port housing 1.0 to 3.0 mm, creating a dead space in all cases visualized.” This “dead space” within the reservoirs of the ports created a place that could collect and hold thrombused blood and/or drug residuals. The study confirmed the problem and stated “[d]eposits of blood was visualized in the dead space of each of the port housings. It is conceivable that these deposits act as a ball valve when aspiration of blood or fluid from the CVC (central venous catheter) is attempted. This is especially true of implanted ports because an inserted needle may have its tip positioned within a buildup of blood products in the base of the port housing.” The study also identified the cause of partial withdrawal occlusion by stating “[t]here is evidence that infusions of blood and blood products, and aspiration of blood . . . can cause a gradual buildup (residue) of blood products that adheres to the wall of some CVC's and collects in the dead space of most subcutaneously implanted ports. Over time, these residues may prevent aspiration while still allowing fluid or drug infusions.”

When the reservoir of an implantable port becomes occluded from the buildup of thrombused blood and/or drug residuals (partial withdrawal occlusion), the clinician will attempt to restore patency with the infusion of a costly lysing agent (TPA, urokinase, etc.). If port patency is not restored after using a lysing agent, it can no longer be safely utilized for chemotherapy and must be surgically removed and a new port implanted. This expensive, painful and potentially dangerous surgical procedure could be avoided if the reservoir of the port could be more effectively flushed after each use.

Partial withdrawal occlusion caused by the buildup of sludge in a port reservoir is a serious problem occurring somewhat frequently. While a significant problem in terms of nursing time, expense and patient discomfort, an even bigger problem caused by the buildup of sludge is the problem of port infections. Infections in implanted ports have been documented to occur rather frequently. Chemotherapy patients are frequently immuno-supressed and/or immuno-compromised and a port infection carries a high risk of patient morbidity and/or mortality. Because ports are a “closed system” that are implanted subcutaneously, port infections are difficult and expensive to try to resolve.

The pathogenesis of port infections has been well documented. Most port infections are caused by coagulase-negative Staphylococcus (especially staphylococcus epidermidis which are the predominant organisms found on the skin). Other strains of bacteria and fungi have also been cultured in explanted ports. These organisms are usually benign in the blood stream in small amounts in a healthy patient because the bloodstream's defenses can combat these bacteria. Once the bacteria exceed about 15 or more colony forming units (CFU's) in the body, the patient is considered bacteremic with a fever and an elevated white blood cell count. In oncology patients who are already immuno-supressed and/or immuno-compromised, this can be a life threatening condition requiring immediate antibiotic therapy and can frequently lead to emergency hospitalization and surgical removal of the infected port.

Therefore, there is a need for a port that minimizes the potential for collecting thrombused blood and/or drug residuals within the reservoir and maximizes the cleaning effectiveness of the flushing solution after the port has been used.

### BRIEF SUMMARY

The present invention is directed to an implantable port for use in the withdrawal and/or delivery of a fluid to or from a patient. The port has a body portion with a generally curved shaped reservoir. A septum extends across the reservoir and a stem extends from the reservoir to an external position. A fluid passageway is formed from the reservoir through the stem and adjacent the septum.

According to another aspect of the invention, a port is provided having a body portion with an internal reservoir formed by a reservoir wall. The reservoir wall is characterized by an ever decreasing radius of curvature extending from adjacent a septum to a bottom of the reservoir. The septum extends across the reservoir. A stem extends from the reservoir to an external position and forms a fluid passageway therein located directly adjacent the septum.

According to other aspects of the invention, methods are provided such as implanting the port subcutaneously in the chest of a patient with the port being oriented such that at least a portion of the stem extends downward when the patient is in a vertical position.

### BRIEF DESCRIPTION OF THE DRAWINGS

FIG. 1 is a perspective view of a port according to a first embodiment of the present invention.

FIG. 2 is a top view of the port illustrated in FIG. 1.

FIG. 3 is a cross-section of the port of FIG. 1 taken along the lines 3-3 of FIG. 2.

FIG. 4 is the cross-section of FIG. 3 with a needle shown inserted into the port and representative fluid flow paths illustrated.

FIG. 5 is a top view of a second embodiment of a port in accordance with the present invention.

FIG. 6 is a cross-section of a third embodiment of a port in accordance with the present invention.

FIG. 7 is an illustration of an implanted port and catheter providing access to the superior vena cava of a heart of a patient.

FIG. 8 is an illustration of an implanted port and catheter providing access to the brain of a patient.

FIG. 9 is an illustration of an implanted port and catheter providing access to the ear of a patient.

FIG. 10 is a cross-section of a representative prior art port configuration.

### DETAILED DESCRIPTION OF THE DRAWINGS AND THE PRESENTLY PREFERRED EMBODIMENTS

The preferred embodiments are described herein in the context of implanted ports, generally. The port of the present invention has many diverse applications known to those of ordinary skill in the art beyond the specific applications disclosed herein. In particular, the port of the present invention is useful for the infusion or withdrawal of a wide range of fluids for a patient. As used herein, the term “fluid” should be interpreted broadly to include infused medicines such as antibiotics, therapies such as chemotherapy treatments, and other fluids such as blood products, bone marrow transplantation (BMT) fluids, total parenteral nutrition (TPA) fluids and to include bodily fluids such as aspirated venous blood, arterial blood, urine, ascites effusions, pleural cavity effusions and cerebral spinal fluids (CSF).

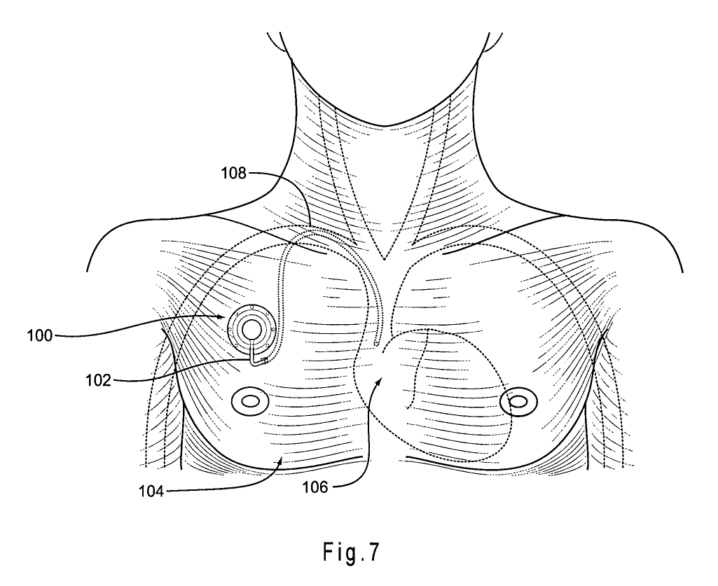

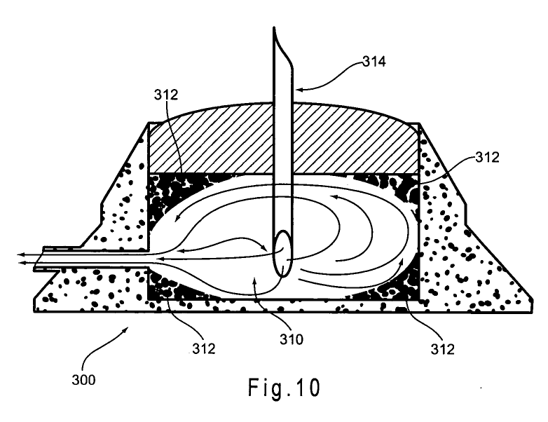

The port of the present invention minimizes the potential for collecting sludge within the reservoir and maximizes the cleaning effectiveness of the flush solution, e.g., a normal saline flush, after the port has been used. In addition, the port of the present invention may be implanted in the manner illustrated in FIG. 7 with the outlet stem directed downward when the patient is an a vertical position in order to further prevent the accumulation of sludge. In contrast, a representative example of a prior art port 300 is illustrated in FIG. 10. The port 300 includes a generally rectangular interior reservoir 310 having corners 312 or “dead spaces.” The corners 312 provide a location for the collection of sludge even after the flushing of the port 300 with saline through the needle 314 as illustrated.

The problem with the buildup of sludge inside the reservoir of implanted ports was discussed in the 1993 article “Turbulent Flow and Catheter Residue” by Denise Macklin, BSN, RNC, CRNI (Journal of Vascular Access Nursing, Vol. 3, No. 3). The process is described: “[i]n implanted ports, it has been found that the “sludge” buildup occurs not in the catheter but in the port. This probably occurs because rigid (huber) needles are used to access ports. Fast flushing moves the saline from the tip of the needle straight down the catheter, and the port residue in the periphery around the needle is left untouched. Over time, the access needle may be placed so that the tip invades this residue (sludge) area. The nurse may or may not be able to flush the port, but blood withdrawal is impossible.” Ultimately, the build up of sludge is significant because it can cause infections and/or occlusions of the port that may require surgery to remove the port.

The best way to avoid the risk of patient morbidity and/or mortality caused by a port infection is to eliminate the colonization of bacteria and/or fingi in the reservoir of the port. The reservoir of an implanted port is an ideal incubator within which bacteria can grow. It is well documented that patients who have had their port accessed have become septic within as little as twenty-four hours. In contrast, the port of the present invention has an improved flushing effectiveness that creates a cleaner port reservoir that helps avoid the collection of sludge and the growth of bacteria. The port of the present invention thereby avoids potential costly and painful surgery to have the infected and/or occluded port removed.

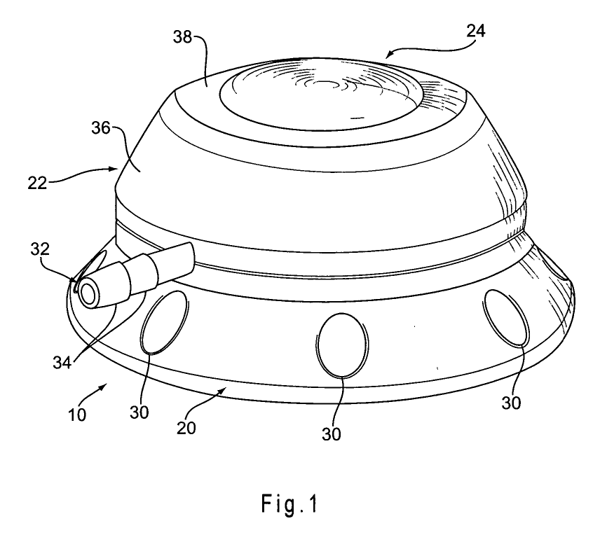

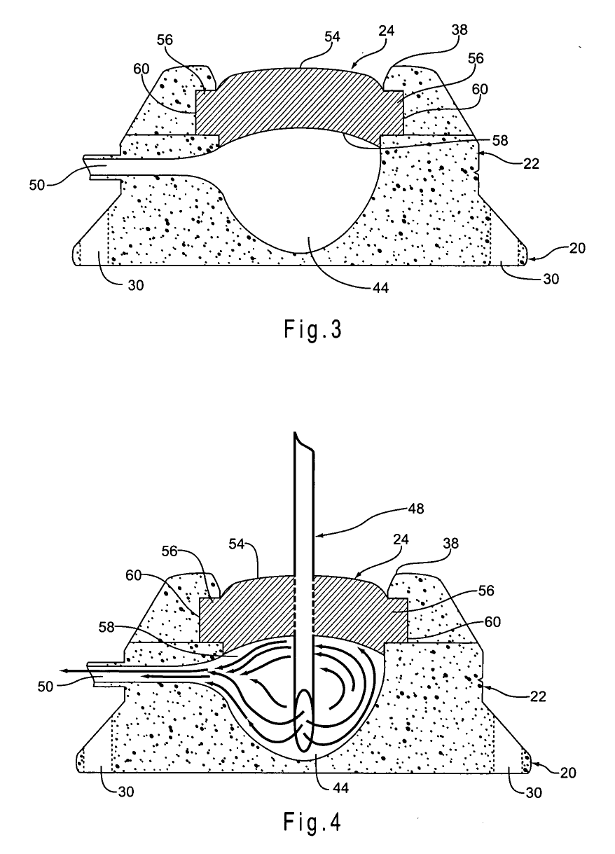

FIG. 1 is an illustration of a port 10, according to a first embodiment of the present invention. The port 10 has a base 20, a central housing 22 and a septum 24. The base 20 includes a plurality of radially spaced apertures 30 that can form suture holes useful to secure the port 10 to the underlying muscle/tissue of a patient during the surgical implantation process, i.e., the port 10 is implanted subcutaneously. An outlet stem 32 extends outward from the central housing 22. The outlet stem 32 includes barbs 34 to provide for a better connection with an associated catheter. While two barbs 34 are illustrated, as few as one barb or greater than two barbs may be used with the port of the present invention. The central housing 22 has an angled outer surface 36 and a needle “kick-in” ring portion 38. The “kick-in” ring portion 38 encircles the septum 24 and creates a larger target area for the clinician during insertion of a needle into the port 10. The septum 24 closes off an interior reservoir 44, as best shown in FIG. 3. The septum 24 is molded from a medical grade silicone that provides access to the interior reservoir 44 through the use of a non-coring needle 48, such as a Huber needle, as illustrated generally in FIG. 4.

The base 20 and the central housing 22 may be formed from a wide range of biocompatible materials know to those of ordinary skill in the art such as titanium or medical grade plastic such as Delrin™. The interior reservoir 44 may be polished to create a surface that helps prevent the collection of sludge. In addition, the interior reservoir 44 may be plated with gold or other known materials to further improve the surface characteristics. In the preferred embodiments, the interior reservoir 44 and the outlet stem 32 are superfinished to meet the ANSI B46 standard of 2-8 microinches of roughness. In the illustrated embodiment, the port 10 is formed from four main interconnected elements: upper and lower housing portions, a septum, and an outlet stem attached thereto.

The structure of the septum 24, the interior reservoir 44 and fluid passageway 50 are best illustrated in FIGS. 3 and 4. The septum 24 has a top surface 54, side portions 56 and a bottom surface 58. The top surface 54 has a slightly curved surface that extends between the “kick-in” ring portions 38 of the central housing 22. The side portions 56 mate within the cutout portions 60 of the central housing 22. The bottom surface 58 forms a slightly concave shape. The concave shape of the bottom surface 58 is useful because it eliminates the corners and dead spaces found inside of conventional port reservoirs. By eliminating the corners and dead spaces inside of the port reservoir the potential for the collection of sludge is greatly reduced.

The interior reservoir 44 has a generally curved or hemispheric shape having a continuously curved interior wall without interruption. The shape of the interior reservoir 44 is generally cornerless and designed to avoid the creation of pockets or dead spaces where sludge may accumulate. A fluid passageway 50 extends from an upper portion of the interior reservoir 44 and adjacent the bottom surface 58 of the septum 24.

The location of the fluid passageway 50 is important because it provides for a greater flushing effectiveness as illustrated in the fluid flow diagram of FIG. 4. The location of the fluid passageway 50 adjacent the septum 24 results in the flushing solution having to pass generally around the entire surface area of the interior reservoir 44 and the bottom surface 58 of the septum 24. As a result, the flushing solution more effectively cleans the interior reservoir 44.

The size of the interior reservoir 44 is also important because it is substantially reduced in volume, 0.4 cc in the illustrated embodiment, as compared to prior art ports with comparably sized septums. The port 10 of the present invention has an increased cleaning effectiveness and reduces the potential for sludge accumulation within the port 10 because it has a greater “clearance factor” than ports of comparable sizes given the use of a standard 10 cc saline flush.

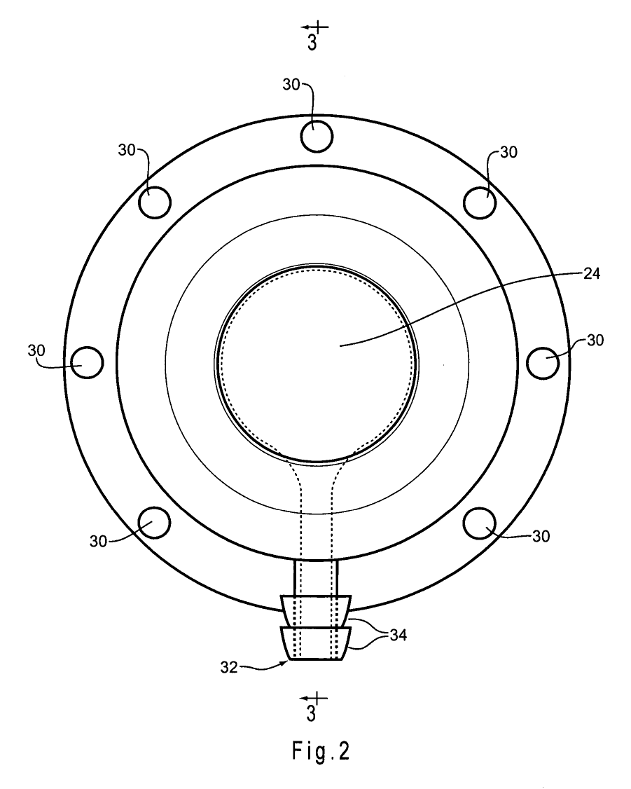

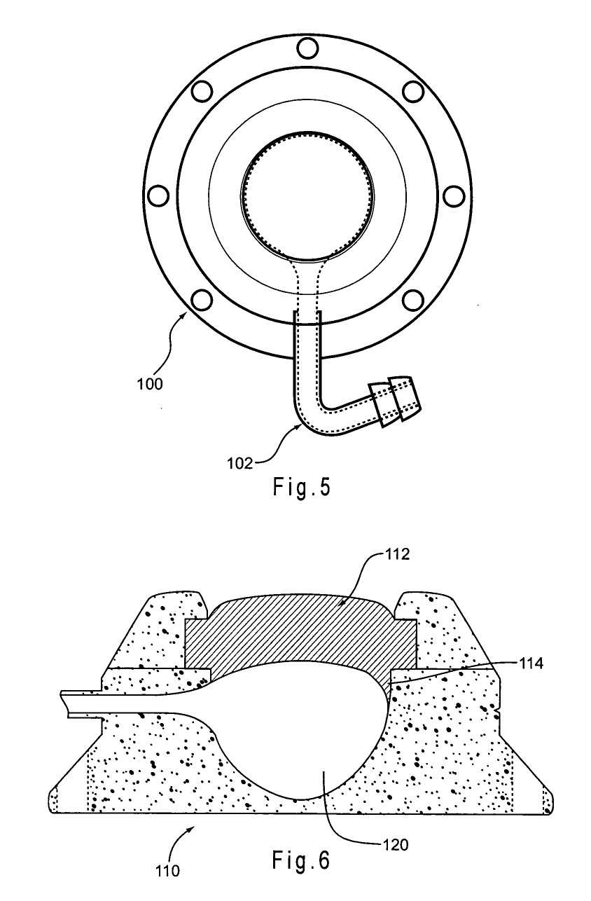

FIGS. 5-6 illustrate second and third embodiments 100, 110 of the port of the present invention. The ports 100, 110 operate in essentially the same manner as the port 10 of FIGS. 1-4. However, with respect to the port 100, the outlet stem 102 includes an upward angled portion. With reference to FIG. 7, the port 100 is useful for placement in the chest 104 of a patient in order to provide access to the superior vena cava 106 of the heart of a patient through the catheter 108. The catheter 108, in this and the other embodiments, may be a conventional catheter as known to those of ordinary skill in the art or may be the catheter disclosed in copending application entitled “High Flow Diffusion Catheter” filed in the name of James Schneiter on Feb. 22, 2005 and bearing Ser. No. 11/063,198. The orientation of the port 100, particularly the downward direction of outlet stem 102, is important because it provides for a more effective cleaning of the port 100 when the patient is in a vertical position. In particular, the pull of gravity on any fluid and/or sludge in combination with the downward direction of the outlet stem 102 helps clean out the interior reservoir by more readily directing sludge outward through the associated catheter during the flushing process. The port 100 is useful for the delivery of medications to the blood stream of a patient or the delivery or withdrawal of blood to or from a patient. With reference to FIG. 6, the port 110 includes a modified septum 112 having a protruding corner portion 114 that further provides a cornerless interior reservoir 120 that helps prevent the accumulation of sludge.

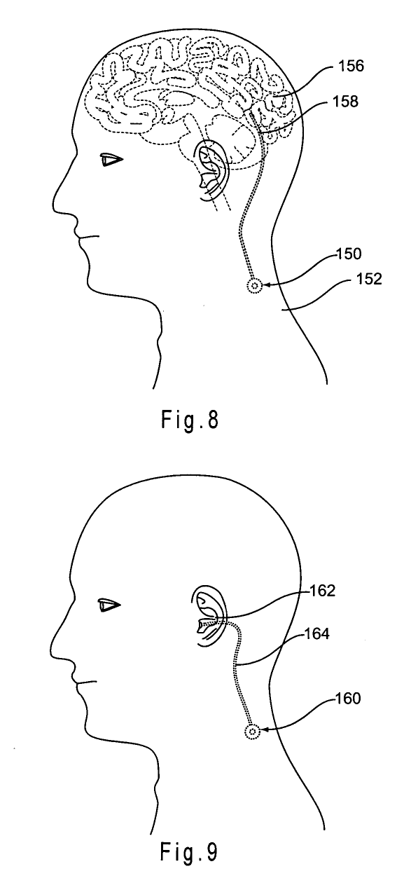

With reference to FIG. 8, the port 150 of the present invention may be implemented for withdrawal of bodily fluids, such as CSF, from the brain. The port 150 may be implanted in the neck 152 area of patient 154 to provide access to the brain 156 through the catheter 158. The port 160 may also be implanted in the same general area as that shown in FIG. 9 to provide access to the ear canal 162 of a patient as illustrated in FIG. 9 through the use of the catheter 164. With respect to both FIGS. 8 and 9, the associated catheter is tunneled subcutaneously such that the terminal end is located adjacent the area of treatment (withdrawal and/or delivery of the fluids). A huber needle is inserted subcutaneously into the port to withdraw or deliver the fluid through the fluid path defined by the needle, port and catheter.

The embodiments described above and shown herein are illustrative and not restrictive. The scope of the invention is indicated by the claims rather than by the foregoing description and attached drawings. The invention may be embodied in other specific forms without departing from the spirit of the invention. For example, the size and external shape and exact construction of the four pieces that form the port (the upper and lower portions of the port housing, the septum and the outlet stem) may be designed in a manner other than as specifically illustrated in the figures. Accordingly, these and any other changes which come within the scope of the claims are intended to be embraced herein.

I claim:

1. An implantable port for use in the withdrawal and/or delivery of a fluid to a patient, said port comprising:
a) a body portion having a generally hemispheric shaped reservoir;
b) a septum extending across the reservoir; and
c) a stem extending from the reservoir to an external position and forming a fluid passageway therein, the fluid passageway being located adjacent the septum.

2. The port of claim 1 wherein the stem includes an intermediate portion and an end portion, the intermediate portion and the end portion forming an angle between 45-135 degrees.

3. The port of claim 2 wherein the intermediate portion and the end portion form an angle of about 75 degrees.

4. The port of claim 3 wherein the reservoir has a generally continuous uninterrupted surface.

5. The port of claim 3 wherein the stem includes a conical-shaped throat portion.

6. The port of claim 3 wherein the reservoir has a polished surface coating.

7. The port of claim 6 wherein the reservoir has a plated surface coating.

8. The port of claim 3 wherein the septum includes a concave bottom portion.

9. The port of claim 8 wherein the septum has a curved top portion.

10. A port for use in the withdrawal and/or delivery of a fluid to a patient, the port comprising:
a) a body portion having an internal reservoir formed by a smoothly curved interior wall;
b) the septum extending across the reservoir; and
c) a stem extending from the reservoir to an external position and forming a fluid passageway therein, the fluid passageway being located directly adjacent the septum.

11. The port of claim 10 wherein the stem includes an intermediate portion and an end portion, the intermediate portion and the end portion forming an angle between 45-135 degrees.

12. The port of claim 11 wherein the intermediate portion and the end portion form an angle of about 75 degrees.

13. The port of claim 12 wherein the reservoir has a generally continuous uninterrupted surface.

14. The port of claim 12 wherein the stem includes a conical-shaped throat portion.

15. The port of claim 14 wherein the reservoir has a polished surface coating.

16. The port of claim 15 wherein the reservoir has a plated surface coating.

17. The port of claim 13 wherein the septum includes a concave bottom portion.

18. The port of claim 17 wherein the septum has a curved top portion.

19. A port for use in the withdrawal and/or delivery of a fluid to a patient having an increased flushing effectiveness, the port comprising:
a) a body portion having an internal reservoir, the reservoir characterized by a generally continuous curved surface and having a bottom;
b) a septum extending across the reservoir; and
c) a stem extending from the reservoir to an external position and forming a fluid passageway therein, the fluid passageway being located at a position substantially spaced apart from the bottom of the reservoir.

20. The port of claim 19 wherein the stem includes an intermediate portion and an end portion, the intermediate portion and the end portion forming an angle between 45-135 degrees.

21. The port of claim 20 wherein the intermediate portion and the end portion form an angle of about 75 degrees.

22. The port of claim 21 wherein the reservoir has a generally continuous uninterrupted surface.

23. The port of claim 22 wherein the stem includes a conical-shaped throat portion.

24. The port of claim 23 wherein the reservoir has a plated and polished surface coating.

25. The port of claim 24 wherein the septum includes a concave bottom portion.

26. A method for flushing a port for use in the withdrawal and/or delivery of a fluid to a patient, the method comprising:
a) providing a syringe of a flushing solution with a non-coring needle;
b) providing a port having an internal reservoir, a septum extending over the reservoir, and a fluid passageway extending from the reservoir to an external position from a location adjacent the septum and forming a fluid passageway therein, the internal reservoir having a volume of 0.4 cc or less; and
c) infusing the flushing solution from the syringe and through the needle into the reservoir in order to flush the reservoir of the port.

27. A method for flushing a port for use in the withdrawal and/or delivery of a fluid to a patient, the method comprising:
a) providing a syringe of a flushing solution with a non-coring needle;
b) providing a port having an internal reservoir with a generally smooth curved surface having a bottom, a septum extending over the reservoir, and a stem extending from the reservoir to an external position and forming a fluid passageway therein, the stem being located in a position substantially spaced apart from the bottom of the reservoir; and
c) infusing the flushing solution from the syringe and through the needle into the reservoir in order to flush the reservoir of the port.

28. A method for the implantation of a port for use in the withdrawal and/or delivery of a fluid to a patient, the method comprising:
a) providing a port having an internal reservoir, a septum extending over the reservoir, and a stem extending from the reservoir to an external position and forming a fluid passageway therein, the stem having an intermediate portion and an angled end portion; and
b) implanting the port subcutaneously in the chest of a patient, the port being oriented such that at least a portion of the stem extends downward when the patient is in a vertical position.

29. A method for the implantation of a port for use in the withdrawal and/or delivery of a fluid to an ear of a patient using a catheter, the method comprising:
a) providing a port having an internal reservoir, a septum extending over the reservoir, and a stem extending from the reservoir to an external position and forming a fluid passageway therein; and
b) implanting the port subcutaneously adjacent an ear of a patient.

30. A method for the implantation of a port for use in the withdrawal and/or delivery of a fluid to a brain of a patient using a catheter, the method comprising:
a) providing a port having an internal reservoir, a septum extending over the reservoir, and a stem extending from the reservoir to an external position and forming a fluid passageway therein; and
b) implanting the port subcutaneously adjacent the brain of a patient and connecting the port to the catheter, the catheter having terminal end located in the brain of the patient.
